(19) United States
(12) Patent Application Publication
(10) Pub. No.: US 2011/0027396 A1
(43) Pub. Date: Feb. 3, 2011

(54) NOVEL MILK THISTLE EXTRACT, METHOD FOR THE PRODUCTION, AND USE

(75) Inventors: Astrid Nagell, Hamburg (DE); Jaime Xiol Aguirre, Barcelona (ES); Santiago Rull Prous, Barcelona (ES)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: EUROMED SA, Mollet De Valles (ES)

(21) Appl. No.: 12/810,178

(22) PCT Filed: Dec. 23, 2008
(86) PCT No.: PCT/DE08/02117

§ 371 Date: Sep. 20, 2010

(30) Foreign Application Priority Data
Dec. 23, 2007 (DE) 10 2007 063 115.6
Aug. 23, 2008 (DE) 10 2008 039 271.5

(51) Int. Cl. A61K 36/28; A61P 1/16; A61P 31/14

(52) U.S. Cl. 424/764

(57) ABSTRACT

A method for preparing a milk thistle fruit extract, in particular a flavanolignan preparation, has an increased release rate and improved absorbability. A pharmaceutical preparation contains the extract and is used, in particular for the treatment and prevention of liver diseases.

### CROSS-REFERENCE TO RELATED APPLICATIONS

This application is a Section 371 of International Application No. PCT/DE2008/002117, filed Dec. 23, 2008, which was published in the German language on Jul. 2, 2009, under International Publication No. WO 2009/080006 A2 and the disclosure of which is incorporated herein by reference in its entirety.

### BACKGROUND OF THE INVENTION

The present invention relates to a method for preparing a milk thistle fruit extract, in particular a flavanolignan preparation having an increased release rate and improved absorbability, and use thereof, in particular for the treatment and prevention of liver diseases.

The milk thistle (Silybum marianum or Carduus marianus) is a plant which is cultivated in particular in southwest and central Europe (Austria, Hungary), and which has become naturalized in Eurasia, North America, South America, and Australia. Production areas are also found in China.

The efficacy of the drug from milk thistle (seeds and fruit) in the treatment and prevention of various forms of liver and gall bladder dysfunction is known. The drug is composed of the ripe fruit from which the pulp has been removed, having a minimum sylimarin content of 1.5% (Pharmacopoea Europaea (Ph. Eur.), 2007). Tinctures (usually alcoholic extracts of the drug) made from milk thistle have been known since ancient times. Isolated silymarin is particularly suitable (for example, DE 1 923 982, DE 1 767 666 (Madaus)).

Silymarin is a flavanolignan complex, i.e., polyhydroxyphenylchromanones, and was first isolated from the plant in the 1960s (Dissertation, Janiak Bernhard, June 1960, Berlin University of Applied Sciences (DE 2020407), Pelter A., Hänsel R., Tetrahedron Letters, 25, (1968)).

Silymarin is composed of a mixture of the flavanolignan complex I-IV; specifically, its primary components are the four flavanolignans: silybin (silybinin) (silymarin I), silydianin (silymarin II), and silychristin (silymarin III)

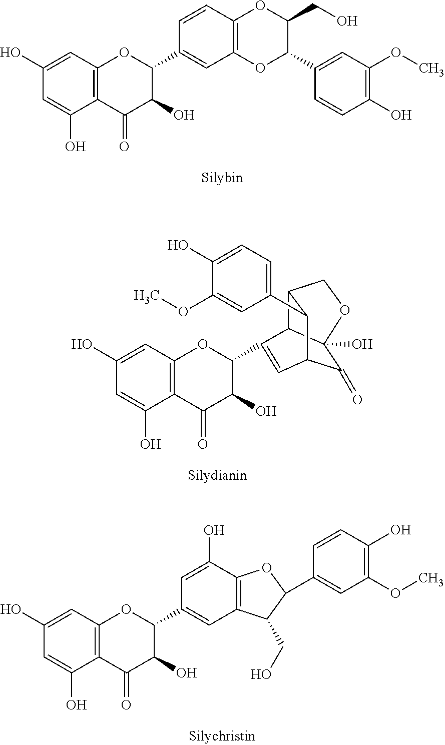

as well as isosilibin (silymarin IV). In these flavolignans the taxifolin is linked to coniferyl alcohol.

Further known secondary components are dehydrosilybin, 3-desoxysilychristin, desoxysilydianin (silymonin), silyadrin, silybinom, silyermin, and neosilymerin.

The fruit of the milk thistle is used for preparing the extract. Such extracts from milk thistle and methods for preparing same have previously been described in the prior art, for example as disclosed in DE 1 923 982, DE 29 14 330 (Madaus).

Also known is a dried extract of milk thistle fruit (Extr. cardui mariae fruct. siccum) which is obtained from the plant drug using, among others, the extraction agent ethyl acetate, and standardized in accordance with the applicable Ph. Eur.

The stated requirements for a dry extract are a content of preferably 30-65% by weight silymarin (other content ranges are possible), the silymarin portion containing the following fractions:

40-65% by weight:

Silibin(in) A and B (diastereomeric mixture, C₂₅H₂₂OH₁₀, MW 482.4) and

10-20% by weight

Isosilibinin A and B (diastereomeric mixture, C₂₅H₂₂OH₁₀, MW 482.4) and

20-45% by weight:

Silidanin and silicristin (C₂₅H₂₂OH₁₀, MW 482.4).

For preparation of an extract, the raw material (in this case, the plant drug) is usually degreased, extracted, filtered, concentrated, and purified.

For said continuous extraction, using ethyl acetate/ethanol/acetone/methanol (optionally in aqueous form) or aqueous mixtures with the above-referenced solvents, filtration is usually performed, followed by concentration. Purification is then carried out using ethanol and hexane (further degreasing), thus obtaining the above-referenced content of silymarin.

Such a composition allows a silymarin release rate of 30 to approximately 40% (measured in accordance with Ph. Eur. 5.7; 2.9.3 (01/2006:20903 as amended, for example using the basket method, paddle model).

However, there is a great need for increasing the release rate of silymarin in the native extract.

It is known that these flavanolignans have little or no solubility in water (the solubility of pure silymarin is approximately 0.08 mg/mL at pH 6.9). Because of this solubility characteristic the release rate of these compounds, and de facto their bioavailability/absorbability in the body of humans or mammals, is inadequate.

In order to increase the release rate, attempts have been made to derivatize the flavanolignans, using polyalcohols, amino sugars, or esters, for example, or to complex them using inclusion compounds such as cyclodextrin (EP 0 422 497 B1 (Madaus)), or using complexing compounds, for example phosphatidylcholine.

However, it is disadvantageous that physiological foreign substances may arise which cause adverse side effects.

It is also known from the prior art that the release rate may be increased by use of carrier substances such as 1-vinyl-2-pyrrolidone, mannitol, and others (EP 0 722 918 B1, U.S. Pat. No. 5,906,991 (Madaus)). In addition, wetting agents such as polysorbates (tensids) are necessary. EP 1 021 198 B1 (Madaus) discloses a silymarin coprecipitate with the use of PEG. However, these referenced methods all have the disadvantage that dosing is made more difficult, and foreign substances may arise which have imprecisely defined side effects.

The object, therefore, is to provide an improved milk thistle fruit extract, in particular one having an advantageously improved silymarin release rate while maintaining the native character. The aim is to prepare the extract essentially without additives, supplements, carrier substances, or wetting agents.

### BRIEF SUMMARY OF THE INVENTION

The object is achieved by providing a method for preparing a milk thistle fruit extract, whereby in the following steps:

a.) The plant drug is extracted with a solvent having moderate polarity (for example, ethyl acetate, ethanol, acetone, methanol, optionally containing aqueous fractions), preferably at 40-80 degrees Celsius, particularly preferably 50-70 degrees Celsius,
b.) separated, preferably filtered,
c.) concentrated, preferably under vacuum with stirring, at a temperature less than 60 degrees Celsius, preferably less than 40 degrees Celsius, and c′) optionally washed with hot water,
d.) combined with ethanol, preferably 96% ethanol or greater, or a solvent of similar polarity, and then combined with hexane or a solvent of similar polarity and concentrated, preferably at a pressure of 1-100 mbar, and the resulting ethanol-water phase is removed,
e.) dried and optionally comminuted,
f.) taken up in anhydrous alcohol, preferably ethanol,
g.) optionally filtered and concentrated, and
h.) dried and optionally comminuted.

The invention further relates to a method for preparing a milk thistle fruit extract having a silymarin release rate of 80% or greater, wherein an extract having a silymarin content of 15-85% by weight, in particular 30-65% by weight, is taken up in anhydrous alcohol, optionally filtered and concentrated, and then dried and optionally comminuted.

The invention therefore relates to a novel milk thistle fruit extract or flavanolignan preparation having an essentially amorphous crystal modification (see comparative tests of the X-ray structural analysis in FIG. 1).

The invention further relates to a medicament composed of a milk thistle fruit extract according to the invention, or use thereof for treatment and prevention of liver and gall bladder dysfunction, in particular for toxic liver damage (fatty liver, alcohol), hepatoses such as mushroom poisoning, acute liver failure, liver necrosis, liver dystrophy, cirrhosis of the liver, hepatic fibrosis, hepatomegaly, and fatty liver degeneration, liver insufficiency, and hepatitis, in particular hepatitis C.

The invention further relates to a pharmaceutical formulation containing a medicament according to the invention composed of a milk thistle fruit extract according to the invention.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 is a graph that shows the altered crystal modification, based on a comparison of silibinin Ch.-B.: 194051 and silibinin Ch.-B.: 7085i, using X-ray structural analysis (conditions corresponding to Example 2);

FIG. 2 is a graph that illustrates the results obtained (conditions corresponding to Example 2) in the complete angular measurement range for Sample Ref. 7233i, using the anhydrous alcohol extraction step f.) of the invention, where the sample mixtures composed of crystalline and amorphous phases were used; and

FIG. 3 is a graph that illustrates the results obtained (conditions corresponding to Example 2) in the complete angular measurement range for Sample Ref. 7232i, not using the anhydrous alcohol extraction step f.) of the invention, where the sample mixtures composed of crystalline and amorphous phases were used.

### DETAILED DESCRIPTION OF THE PREFERRED EMBODIMENTS OF THE INVENTION

As noted above, one aspect of the present invention is method for preparing a milk thistle fruit extract, whereby in the following steps:

a.) The plant drug is extracted with a solvent having moderate polarity (for example, ethyl acetate, ethanol, acetone, methanol, optionally containing aqueous fractions), preferably at 40-80 degrees Celsius, particularly preferably 50-70 degrees Celsius,
b.) separated, preferably filtered,
c.) concentrated, preferably under vacuum with stirring, at a temperature less than 60 degrees Celsius, preferably less than 40 degrees Celsius, and c′) optionally washed with hot water,
d.) combined with ethanol, preferably 96% ethanol or greater, or a solvent of similar polarity, and then combined with hexane or a solvent of similar polarity and concentrated, preferably at a pressure of 1-100 mbar, and the resulting ethanol-water phase is removed,
e.) dried and optionally comminuted,
f.) taken up in anhydrous alcohol, preferably ethanol,
g.) optionally filtered and concentrated, and
h.) dried and optionally comminuted.

Surprisingly, the additional step f.) results in a significant increase in the silymarin release rate to 80% (see comparative examples). This is particularly advantageous, since a lower dosage of the milk thistle fruit extract according to the invention is achieved. It is also advantageous that a quality is attained which in the prior art is achievable only using additives, supplements, carrier substances, and wetting agents.

The term “anhydrous alcohol” in step f.) preferably includes C1-C4 alcohols, particularly preferably ethanol, such as 99% or even 99.5% pure.

Therefore, the invention further relates to a method for preparing a milk thistle fruit extract having a silymarin release rate of 80% or greater, wherein an extract having a silymarin content of 15-85% by weight, in particular 30-65% by weight, is taken up in anhydrous alcohol, optionally filtered and concentrated, and then dried and optionally comminuted.

Within the scope of the present invention, “silymarin” refers to a substance mixture containing (at least) the four substances silybin, silydianin, silychristin, and isosilbin in various concentrations. The ratios of these substances with respect to one another, and the presence of additional substances in the mixture, are not important. However, it is preferable that these substances meet the requirements of Ph. Eur. or DAB as amended. This is the case for the present invention.

A “silymarin release rate of 80% or greater” means that the active substances are at least 80% soluble in aqueous solution (standard according to Ph. Eur.; see examples).

This advantageously results in improved absorbability.

The milk thistle fruit extract obtained is particularly suitable, in that as the result of method step f.) the crystalline fractions in the resulting extract are significantly reduced, and a milk thistle fruit extract having an essentially amorphous crystal modification is obtained.

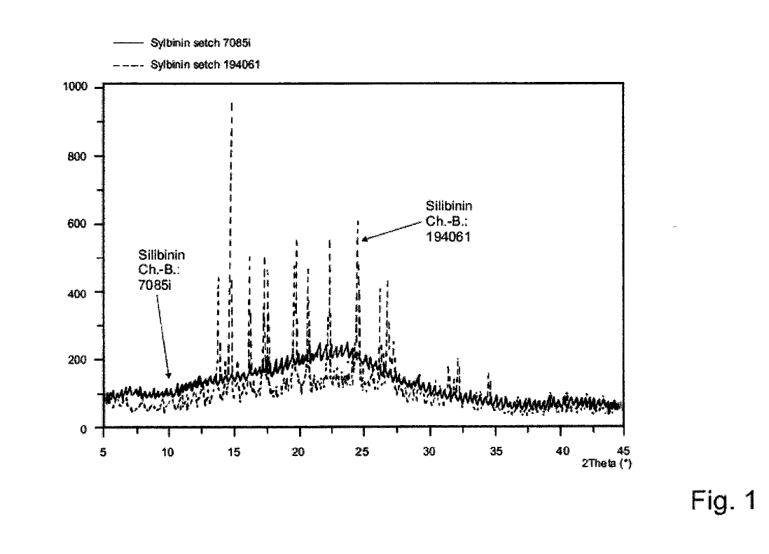

The invention therefore relates to a novel milk thistle fruit extract or flavanolignan preparation having an essentially amorphous crystal modification (see comparative tests of the X-ray structural analysis in FIG. 1).

In one particularly preferred embodiment, the invention relates to a novel milk thistle fruit extract or flavanolignan preparation composed of an amorphous crystal modification, wherein the crystalline fraction is less than 20%, preferably less than 10%, particularly preferably less than 7%, even 5%.

The invention therefore further relates to a medicament composed of a milk thistle fruit extract according to the invention, or use thereof for treatment and prevention of liver and gall bladder dysfunction, in particular for toxic liver damage (fatty liver, alcohol), hepatoses such as mushroom poisoning, acute liver failure, liver necrosis, liver dystrophy, cirrhosis of the liver, hepatic fibrosis, hepatomegaly, and fatty liver degeneration, liver insufficiency, and hepatitis, in particular hepatitis C.

The invention further relates to a pharmaceutical formulation containing a medicament according to the invention composed of a milk thistle fruit extract according to the invention.

The milk thistle fruit extracts according to the invention may be provided in the form of pharmaceutical preparations in dosage units. This means that the preparation [may] be present in the form of individual portions, for example tablets, dragees, capsules, pills, suppositories, and ampoules, the active substance content of which [may] correspond to a fraction or a multiple of a single dose. The dosage units may contain, for example, 1, 2, 3, or 4 single doses, or ½, ⅓, or ¼ of a single dose. A single dose preferably contains the quantity of active substance which is dispensed in one administration, and which typically corresponds to a whole daily dose or a half, third, or fourth of a daily dose.

Nontoxic, inert, pharmaceutically suitable carrier substances are understood to mean solid, semisolid, or liquid diluents, fillers, and formulation adjuvants of all types.

Tablets, dragees, capsules, pills, granules, suppositories, solutions, syrups, suspensions, and emulsions are named as preferred pharmaceutical formulations. Tablets, dragees, capsules, pills, and granules may contain the active substance or substances in addition to the customary carrier substances, such as a) fillers and extenders, for example starches, lactose, sucrose, glucose, mannite, and silicic acid, b) binders, for example carboxymethylcellulose, alginates, gelatins, and polyvinylpyrrolidone, c) humectants, for example glycerin, d) disintegrants, for example agar-agar, calcium carbonate, and sodium carbonate, e) solubility retardants, for example paraffin, f) absorption accelerators, for example quatenary ammonium compounds, g) wetting agents, for example cetyl alcohol and glycerin monostearate, h) adsorbents, for example kaolin and bentonite, and i) lubricants, for example talc, calcium and magnesium stearate, and solid polyethylene glycols, or mixtures of the substances stated under a) through i).

Tablets, dragees, capsules, pills, and granules may be provided with customary coatings and shells optionally containing opacifying agents, and may also have a composition such that they deliver the active substance or substances only in the intestinal tract or preferably in a specific portion thereof, optionally in a delayed manner, wherein polymeric substances and waxes, for example, may be used as encapsulating compounds.

The active substance or substances may also be present in microencapsulated form, optionally with one or more of the above-referenced carrier substances.

In addition to the active substance or substances, suppositories may contain customary water-soluble or water-insoluble carrier substances, for example polyethylene glycols, fats, for example cocoa butter, and higher esters (for example, C14 alcohol with C16 fatty acid), or mixtures of these substances.

In addition to the active substance or substances, solutions and emulsions may contain customary carrier substances such as solvents, solubilizers, and emulsifiers, for example water, ethyl alcohol, isopropyl alcohol, ethyl carbonate, ethyl acetate, benzyl alcohol, benzyl benzoate, propylene glycol, 1,3-butylene glycol, dimethylformamide, oils, in particular cottonseed oil, peanut oil, corn germ oil, olive oil, castor oil, and sesame oil, glycerin, glycerin formal, tetrahydrofurfuryl alcohol, polyethylene glycols, and fatty acid esters of sorbitan, or mixtures of these substances.

In addition to the active substance or substances, suspensions may contain customary carrier substances such as liquid diluents, for example water, ethyl alcohol, and propylene glycol, suspension agents, for example ethoxylated isostearyl alcohols, polyoxyethylene sorbite, and sorbitan esters, microcrystalline cellulose, aluminum metahydroxide, bentonite, agar-agar, and gum tragacanth, or mixtures of these substances. The referenced formulation forms may also contain dyes, preservatives, and fragrance- and taste-enhancing additives, for example peppermint oil and eucalyptus oil, and sweeteners, for example saccharin.

### EXAMPLES

The following examples are used solely to illustrate the invention, without limiting the invention thereto.

### Example 1

### Comparative Tests for Release of Silymarin

Comparative extracts (silibinin Ch.-B.: 194051, Ch.-B.: 7085i) according to the above description were prepared, silibinin Ch.-B.: 7085i being prepared according to additional method step f.).

The following (silymarin) active substance release resulted at pH 7.5, under the conditions stated in Ph. Eur. (Dissolution test of solids; Ph. Eur 5.7; 2.9.3 (01/2006:20903):

| Sample | Sample taken | Dissolved quantity in % |
|---|---|---|
| Silibinin Ch.-B.: 194051 | After 30 min | 4.16% |
| Silibinin Ch.-B.: 7085i | After 30 min | 49.13% |

The results show that treatment with anhydrous ethanol in step f.) causes the previously poorly soluble silibinin mixture, composed of an amorphous and crystalline structure, to be converted to an amorphous crystal modification (see FIG. 1) (i.e., the crystal lattice structure is altered), resulting in improved solubility and active substance release.

This additional method step allows preparation of the above-described extract having an active substance release of at least 80% total silymarin, calculated as silibinin (HPLC—Ph. Eur. 01/2007:2071), after 30 min, since this method improves the solubility not only of the silibinin, but also of the other silymarin isomers.

FIG. 1 shows the altered crystal modification, based on a comparison of silibinin Ch.-B.: 194051 and silibinin Ch.-B.: 7085i, using X-ray structural analysis (conditions corresponding to Example 2).

Radiographic analyses were carried out on an X'Pert Pro MPD diffractometer from PANalytical B.V., using Bragg-Brentano geometry and an X'Celerator detector.

Further comparative tests are described below:

### Example 2

### Methodology

Sample Preparation:

Two Solid Powder Samples of Products

Ref. 7233i with step f.), using the method according to the invention, and

Ref. 7232i without step f.)

X-Ray Diffraction Analysis Using the Powder Technique

Introduction of portions of the powdered material inserted in Lindemann glass capillaries of 0.5 millimeter diameter.

Equipment and test conditions:

PANalytical X'Pert PRO MPD diffractometer with a 9/9 goniometer having a radius of 240 millimeters, parallel lens with hybrid monochromator, and transfer geometry with sample holders for capillaries, with a spinner.

Cu—Ka radiation (k=1.5406 Å).

Power: 45 kV-40 mA.

Slit which at 0.19 millimeters fixes the quantity

Soller aperture with 0.02 radiation in the incident quantity and the diffracted quantity

X'Celerator measuring unit having an active length of 2122.

Flushing 29 of 3 to 60° 29 having a step size of 0.017 and a measurement time of 1500 sec per step.

Objective

Production of X-ray diffraction diagrams using the powder technique. Determination of the rate of crystallization.

Methodology

The rate of crystallization is the weight percentage of the crystalline phase in a sample mixture composed of crystalline and amorphous phases, using a crystallization index Ci:

Ci=100[Xc/(Xa+Xc)], where Xc represents the weight fraction of the amorphous phases.

The values of Xc were determined from the sum of the regions of all narrow points (in the crystalline phase) in the angular range of the study. The values of Xa were obtained by determining the regions of wide points or “halos” (in the amorphous phase).

Results

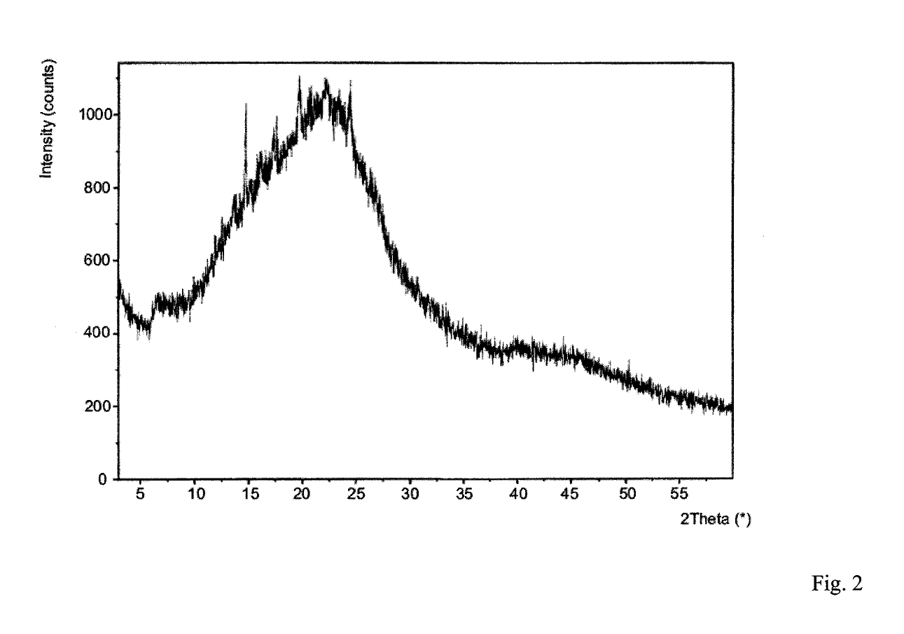

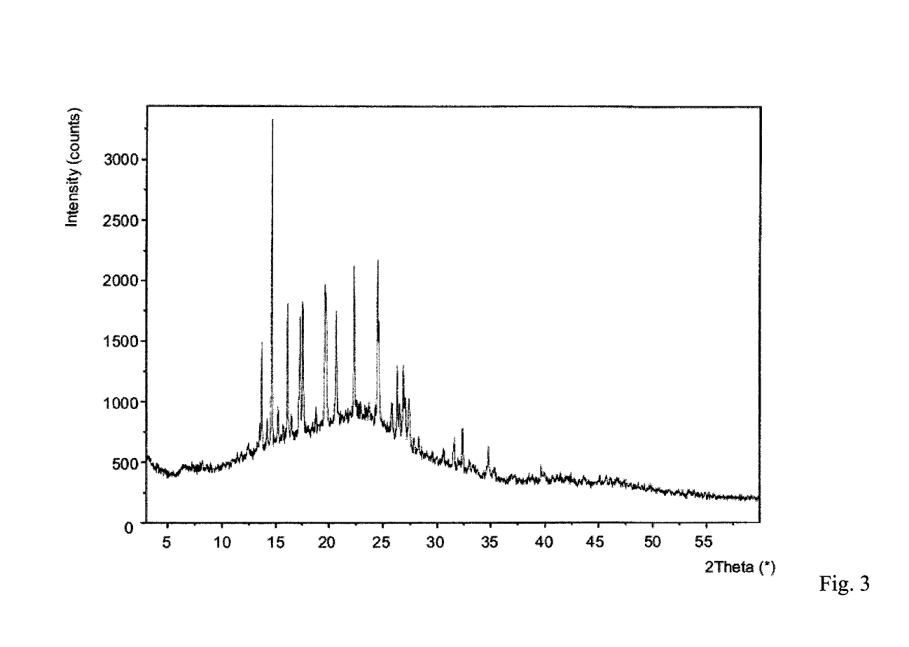

FIGS. 2 and 3 illustrate the diagrams obtained in the complete angular measurement range. Sample mixtures composed of crystalline and amorphous phases were used.

The resulting Ci values were 7% for sample Ref. 7233i (see FIG. 2), and were 24% for sample Ref. 7232i (see FIG. 3).

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1.-9. (canceled)

10. A method for preparing a milk thistle fruit extract having a silymarin release rate of 80% or greater, the method comprising:
(i) providing an extract containing 15-85% by weight of silymarin;
(ii) taking up the extract from (i) in anhydrous alcohol to form an anhydrous alcohol extract;
(iii) optionally filtering the anhydrous alcohol extract from (ii) to form an optionally filtered anhydrous alcohol extract;
(iv) optionally concentrating the anhydrous alcohol extract from (ii) or the optionally filtered anhydrous alcohol extract from (iii) to form an optionally concentrated anhydrous alcohol extract;
(v) drying any one of the anhydrous alcohol extract, the optionally filtered anhydrous extract or the optionally concentrated anhydrous alcohol extract to form a dried extract; and
(vi) optionally comminuting the dried extract.

11. The method of claim 10, wherein the extract of (i) contains 30-65% by weight of silymarin.

12. A method for preparing a milk thistle fruit extract having a silymarin release rate of 80% or greater, the method comprising:
(a) extracting the extract from a milk thistle fruit with a solvent having moderate polarity;
(b) separating the extract from (a);
(c) concentrating the extract from (b) to form a concentrated extract;
(d) combining the concentrated extract with ethanol or a solvent of similar polarity to ethanol to form a water phase of the extract, and then combining the water phase of the extract with hexane or a solvent of similar polarity to hexane to form an organic phase of the extract, followed by concentrating the combined water phase of the extract combined with the organic phase of the extract, and recovering the water phase extract to form the recovered extract;
(e) drying and optionally comminuting the recovered extract to form dried recovered extract;
(f) taking up the dried recovered extract in anhydrous alcohol to form an anhydrous alcohol extract;
(g) optionally filtering and concentrating the anhydrous alcohol extract; and
(h) drying and optionally comminuting the anhydrous alcohol extract.

13. The method of claim 12, wherein:
(a) is performed at a temperature of 50-70 degrees Celsius;
the separating of (b) is performed by filtering;
the concentrating of (c) is performed under vacuum with stirring, at a temperature less than 60 degrees Celsius, and optionally washing the concentrated extract with hot water; and
the combining the concentrated extract (d) is with ethanol to form the water phase of the extract, and then with hexane to form the organic phase of the extract, wherein the concentrating the combined water phase of the extract combined with the organic phase of the extract is performed at a pressure of 1-100 mbar.

14. The method of claim 13, wherein:
(a) is performed at a temperature of 40-80 degrees Celsius and the solvent having moderate polarity is selected from the group comprising ethyl acetate, ethanol, acetone and methanol, optionally containing aqueous fractions;
the concentrating is at a temperature of less than 40 degrees Celsius;
the ethanol of the combining in (d) is at least 96% ethanol; and
the anhydrous alcohol of (f) is an anhydrous C1-C4 alcohol.

15. The method of claim 12, wherein, in (f) the anhydrous alcohol is ethanol.

16. The method of claim 12, wherein in (a), the solvent of moderate polarity is selected from the group comprising ethyl acetate, ethanol and methanol.

17. The method of claim 16, wherein the solvent of moderate polarity is ethyl acetate.

18. Milk thistle fruit extract obtainable by a method according to claim 10.

19. The milk thistle fruit extract according to claim 18, wherein the milk thistle fruit extract consists essentially of an amorphous crystal modification.

20. The milk thistle fruit extract according to claim 18, wherein the milk thistle fruit extract is composed of an amorphous crystal modification having a crystalline fraction less than 20%.

21. The milk thistle fruit extract according to claim 20, wherein the milk thistle fruit extract is composed of an amorphous crystal modification having a crystalline fraction less than 10%.

22. The milk thistle fruit extract according to claim 21, wherein the milk thistle fruit extract is composed of an amorphous crystal modification having a crystalline fraction less than 7%.

23. A pharmaceutical preparation containing a milk thistle fruit extract according to claim 18.

24. A method of treating or preventing liver dysfunction or gall bladder dysfunction in a subject by administering to the subject the milk thistle fruit extract according to claim 18.

25. The method for treating or preventing liver dysfunction according to claim 24, wherein the liver dysfunction is selected from the group comprising toxic liver damage, hepatoses, acute liver failure, liver necrosis, liver dystrophy, cirrhosis of the liver, hepatic fibrosis, hepatomegaly, fatty liver degeneration, liver insufficiency and hepatitis.

26. The method of claim 26, wherein the hepatitis is hepatitis C.

27. Milk thistle fruit extract obtainable by a method according to claim 12.

28. The milk thistle fruit extract according to claim 27, wherein the milk thistle fruit extract consists essentially of an amorphous crystal modification.

29. The milk thistle fruit extract according to claim 27, wherein the milk thistle fruit extract is composed of an amorphous crystal modification having a crystalline fraction less than 20%.

30. The milk thistle fruit extract according to claim 29, wherein the milk thistle fruit extract is composed of an amorphous crystal modification having a crystalline fraction less than 10%.

31. The milk thistle fruit extract according to claim 30, wherein the milk thistle fruit extract is composed of an amorphous crystal modification having a crystalline fraction less than 7%.

32. A pharmaceutical preparation containing a milk thistle fruit extract according to claim 27.

33. A method of treating or preventing liver dysfunction or gall bladder dysfunction in a subject by administering to the subject the milk thistle fruit extract according to claim 27.

34. The method for treating or preventing liver dysfunction according to claim 33, wherein the liver dysfunction is selected from the group comprising toxic liver damage, hepatoses, acute liver failure, liver necrosis, liver dystrophy, cirrhosis of the liver, hepatic fibrosis, hepatomegaly, fatty liver degeneration, liver insufficiency and hepatitis.

35. The method of claim 34, wherein the hepatitis is hepatitis C.
